(19) United States
(12) Patent Application Publication
(10) Pub. No.: US 2026/0074014 A1
(43) Pub. Date: Mar. 12, 2026

(54) COMPUTERIZED DECISION TOOL FOR SARS-COV-2 VARIANTS PREDICTION

(71) Applicant: Pfizer Inc., New York, NY (US)

(72) Inventors: Annaliesa Sybil Anderson, Upper Saddle River, NJ (US); Subrata Saha, Ossining, NY (US); Kena Anne Swanson, Pearl River, NY (US); Qi Yang, Orangeburg, NY (US)

(73) Assignee: Pfizer Inc., New York, NY (US)

(21) Appl. No.: 19/105,275

(22) PCT Filed: Aug. 17, 2023
(86) PCT No.: PCT/IB2023/058249

§ 371 Date: Feb. 20, 2025

Related U.S. Application Data
(60) Provisional application No. 63/399,908, filed on Aug. 22, 2022.

(51) Int. Cl. G16B 20/20; G06N 20/10; G16B 15/30; G16B 30/00; G16B 40/20

(52) U.S. Cl. CPC G16B 20/20; G06N 20/10; G16B 15/30; G16B 30/00; G16B 40/20

(57) ABSTRACT

Technology is disclosed for a method for screening genetic mutations that can be used to predict vaccine composition, the method may include selecting a plurality of genome samples, partitioning the plurality of genome samples into N groups, where N is an integer larger than 1, identifying genomic isolates with phenotypic statuses from each of the N groups of genome samples by training at least one linear support vector machine with the genome samples, the identification of the isolates between each of the N groups of the genomic isolates performed in parallel, and assessing the identified genomic isolates using a performance metric.

### REFERENCE TO RELATED APPLICATIONS

This application is a 371 of international application number PCT/IB2023/058249 filed Aug. 17, 2023, which claims priority to U.S. Patent Application No. 63/399,908 filed on Aug. 22, 2022.

The above-listed application is incorporated by reference in its entirety.

### SUBJECT OF THE INVENTION

The subject matter disclosed herein relates to means and methods to predict virus variants. Specifically, the subject matter is related to using machine learning algorithms to predict future virus variants.

### BACKGROUND OF THE INVENTION

Viral and bacterial respiratory infections, such as influenza, impact a large population every year and have symptoms that range from minimal to severe. Like all viruses, the SARS-CoV-2 virus also evolves over time. Variants of concern (VOCs) have started to emerge since the end of 2020. In less than two years, several variants of concern (VOCs) and variants of interest (VOIs) emerged, including Alpha, Beta, Gamma, Delta, and the recent Omicron variant consisting of up to 39 amino acid changes in the spike glycoprotein.

As VOCs have the potential to increase the transmissibility, resist therapeutics, escape vaccine-induced immunity, and/or cause more severe infection, innovative approach and research are needed to understand the genetics of VOCs. Predicting VOCs can be very crucial for surveillance, early warnings, and timely update the vaccines to match emerging drifting variants that could save thousands of human lives. As such, there exists a need for computational methods to predict SARS-CoV-2 variants accurately and with lead time.

### SUMMARY OF THE INVENTION

This summary is provided to introduce a selection of concepts in a simplified form that is further described below in the detailed description. This summary is neither intended to identify key features or essential features of the claimed subject matter nor to be used in isolation as an aid in determining the scope of the claimed subject matter.

Embodiments of the technologies described in the present disclosure enables a method for screening genetic mutations to produce vaccines, the method may include selecting a plurality of genome samples, identifying genomic isolates with phenotypic statuses from the selected genome samples by training at least one linear support vector machine with the genome samples, and assessing the identified genomic isolates using a performance metric.

In another embodiment, a method for screening genetic mutations that can be used to predict vaccine composition may include selecting a plurality of genome samples, partitioning the plurality of genome samples into N groups, where N is an integer larger than 1, identifying genomic isolates with phenotypic statuses from each of the N groups of genome samples by training at least one linear support vector machine with the genome samples, the identification of the isolates between each of the N groups of the genomic isolates performed in parallel, and assessing the identified genomic isolates using a performance metric.

In yet another embodiment, a computerized system for screening genetic mutations may include one or more processors, and a computer memory having computer-executable instructions stored thereon for performing operations when executed by one or more processors, the operations can include selecting a plurality of genome samples, identifying genomic isolates with phenotypic statuses from the selected genome samples by training at least one linear support vector machine with the genome samples, and assessing the identified genomic isolates using a performance metric.

### BRIEF DESCRIPTION OF THE DRAWING

Aspects of the disclosure are described in detail below with reference to the attached figures, wherein:

FIG. 1 illustrates a block diagram of an exemplary variant prediction algorithm in accordance with the subject matter presented herein;

FIG. 2 illustrates a diagram depicting an exemplary variant sample selection algorithm in accordance with the subject matter presented herein;

FIG. 3 illustratively depicts an exemplary iterative mutation cluster prediction or IMCP algorithm in accordance with the subject matter presented herein;

FIG. 4 illustrates a block diagram of a distributed mutation cluster prediction system in accordance with the subject matter presented herein;

FIG. 5 illustrates an example of a single iteration of a cluster prediction algorithm in accordance with the subject matter presented herein;

FIG. 6 illustrates an example of an iterative predictive cluster inference algorithm in accordance with the subject matter presented herein;

FIG. 7 illustrates an example of a distributed learning framework in accordance with the subject matter presented herein;

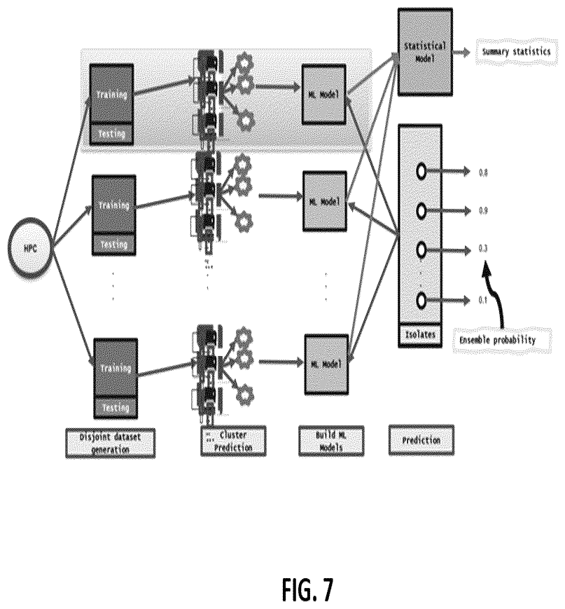

FIG. 8 is a figure illustrating some of the dominate lineages within the Covid-19 Omicron variant predicted by an algorithm in accordance with the subject matter presented herein;

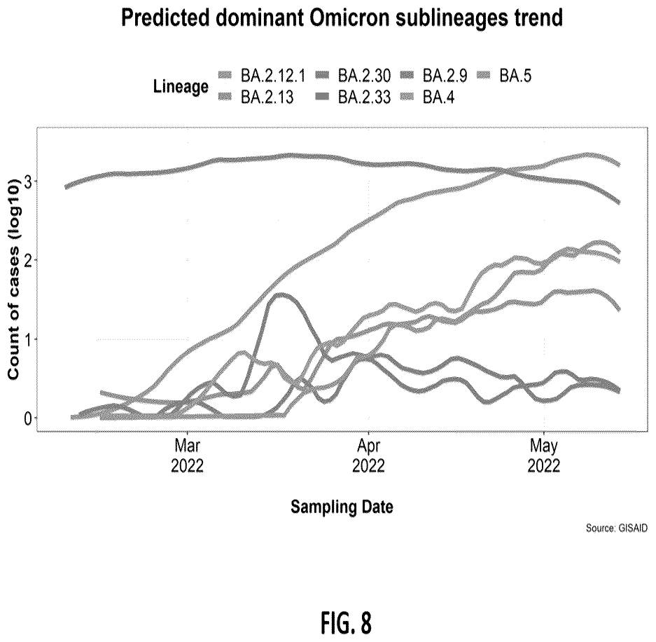

FIG. 9 illustrates one example of the VOC prediction capabilities of the subject matter disclosed herein; and

FIG. 10 illustrates an exemplary application of a prediction algorithm in accordance with the subject matter presented herein.

### DETAILED DESCRIPTION OF THE INVENTION

The subject matter of the present disclosure is described herein with specificity with the help of different aspects to meet statutory requirements. However, the description itself is not intended to limit the scope of this patent. The claimed subject matter might be embodied in other ways, to include different steps or combinations of steps similar to the ones described in this present disclosure, in conjunction with other present or future technologies. Moreover, although the terms “step” and/or “block” may be used herein to connote different elements of methods employed, the terms should not be interpreted as implying any particular order among or between various steps disclosed herein, unless and except when the order of individual steps is explicitly stated. Each method described herein may comprise a computing process that may be performed using any combination of a hardware, firmware, and/or software. For instance, various functions may be carried out by a processor executing instructions stored in a computer memory. The methods may also be embodied as computer-useable instructions stored on computer storage media. The methods may be provided by a stand-alone application, a service or a hosted service (stand-alone or in combination with another hosted service), or a plug-in to another product, to name a few.

Aspects and embodiments of the present disclosure relate to computerized decision support tools for efficiently and accurately detect genome-wide non-spurious protein mutations of SARS-CoV-2 viral genomes. The SARS-CoV-2 virus has impacted a large population and have symptoms that range from asymptomatic to severe. A SARS-CoV-2 infection may trigger respiratory conditions such as respiratory infections or may comprise non-infectious respiratory system symptoms. Although some aspects or embodiments of the subject matter presented herein describe the SARS-CoV-2 viral genome, it is contemplated that such aspects or embodiments may apply to viral genomes in generally.

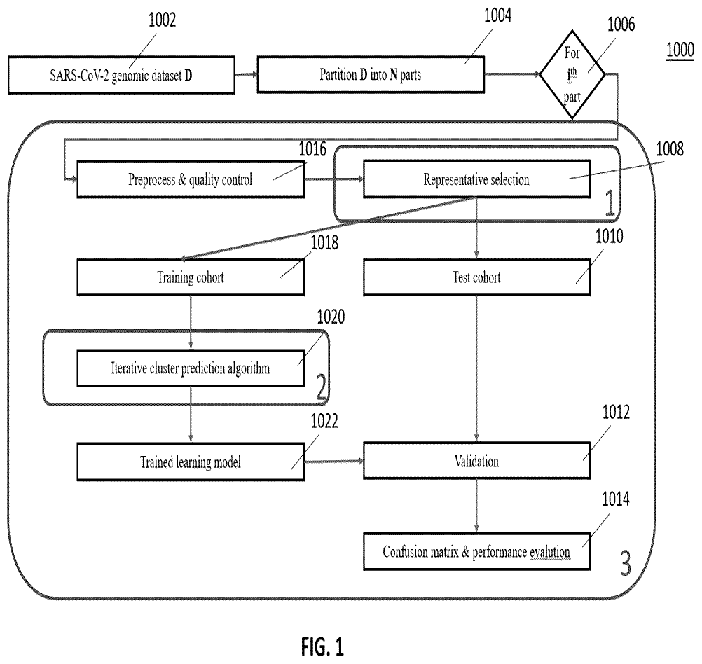

To enable a prediction of SARS-CoV-2 variants, embodiments of the present subject matter provide one or more methods or tools for selecting genome samples. GISAID (https://www.gisaid.org/) provides more than 12 million SARS-CoV-2 genomes. As multiple genomes are coming from the same lineages, many of them are redundant sequence and non-informative. In addition, performing machine learning algorithms on this entire set of genomes will demand an enormous amount of resources. Moreover, the low coverage genomes and incomplete genomes need to be filtered out. As such, a representative selection algorithm may be presented herein to generate a set of high-quality, high coverage and less-noisy representative genomes from each of the lineages of SARS-CoV-2 virus. In one example, referring now to FIG. 1, in step 1002 and 1004, a SARS-CoV-2 genomic data set D may be firstly partitioned into N parts, where N is an integer. And in steps 1006, 1016 and 1008, for any given part from the N parts, the genomic samples from the data set D may be pre-processed and a selection algorithm (e.g., step 1008) may be performed to choose a set of qualified samples. Where using this pre-selected sample set, training cohort (e.g., step 1018) and test cohort (e.g., step 1010) may be constructed. Subsequently, an iterative cluster prediction algorithm may be executed using the constructed training cohort (e.g., step 1020) and trained learning model may be obtained for the prediction algorithm (e.g., step 1022). Furthermore, the test cohort can be used to evaluate the trained model (e.g., step 1012), using performance metrics (e.g., step 1014) and presented in a confusion matrix format.

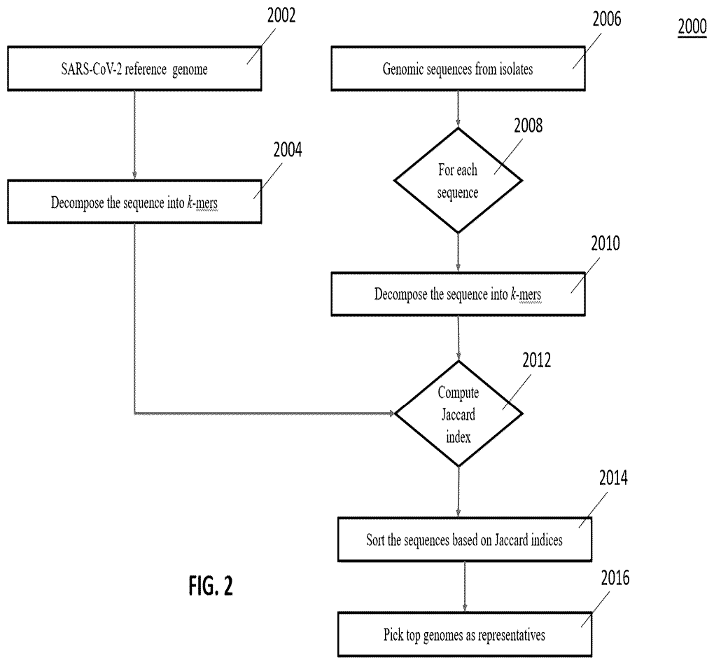

In practice, an iterative cluster prediction algorithm 1020 may be an algorithm similar to what is presented in FIG. 2. Referring now to FIG. 2, in step 2002, one may firstly designate a reference genome (e.g., hCoV-19/Wuhan/WIVO4/2019 (WIV04)) and refers to that reference genome as g for the purpose of illustration. Furthermore, a set (e.g., designated A) of all k-mers from the sequence of the reference genome g may be collected where k-mers constitute of all possible substrings of length k (e.g., k=20), as illustrated in step 2004. Then, following a step 2006 where genomic sequences from isolates are obtained, for each lineage (e.g., step 2008), one may pick those isolates having is_complete and is_high_coverage flags marked as true in GISAID. Where GISAID considers length of genomes>29,000 nucleotide, and where 90% of the genome are complete genomes as is_complete; and where a genome is designated is_high_coverage when a genome has <15 Ns. As such, these sequences may be labeled as high quality (HQ) sequences.

In some embodiments, the k-mers may be used as a proxy to determine representative sequences since the k-mers can indicate low quality in sequences. For example, for each of the HQ sequence g′ in each lineage, one may collect all the k-mers in g′, and designate this set as B. One may then compute the Jaccard index between the A and B sets as illustrated in step 2012. The result may be used to gauge the similarity and diversity of sample sets, as illustrated here, such that more similar g′ is to g, the less noisy g′ will be, where “noisy” or “noise” refer to the spurious mutations that may be sequencing error or low quality of isolates.

$$J ( A , B ) = \frac{\vert A ⋂ B \vert}{\vert A ⋃ B \vert} = \frac{\vert A ⋂ B \vert}{\vert A \vert + \vert B \vert - \vert A ⋂ B \vert} .$$

Furthermore, in step 2014, one may sort the sequences in each lineage in a non-decreasing order based on their Jaccard indices and take a set of top sequences as the representative genomes for that particular lineage as shown in step 2016.

In practice, to predict phenotypic status of an isolate (e.g., whether an isolate is a VOC or not), a machine learning algorithm such as a supervised iterative cluster prediction algorithm 3000 may be utilized to effectively identify a subset of discriminating mutations that can elucidate the phenotypic status. In some embodiments, such cluster prediction algorithm 3000 may be constructed by assemble a set of linear support vector machines or LSVMs. Firstly, a dataset D may be built and formatted, where D may be a matrix D=m×n where m and n are the number of samples and mutations, respectively. In some embodiments, m may be further decomposed into 2 parts, e.g., (1) a set of isolates known to have phenotypic status, such as VOC and (2) another set of isolates known to have different phenotypic status, such as non-VOC, non-VOI, or non-VUM. For the purpose of illustrating the working principles in accordance with the subject matter disclosed herein, one may pick 80% samples from m without replacement that constitute training examples and the rest from test examples for the cluster prediction algorithm. For each sample in m, each of the mutations may be encoded by two numerical values (e.g., 0 and 1) where “0” indicates no mutation and “1” indicates a mutation.

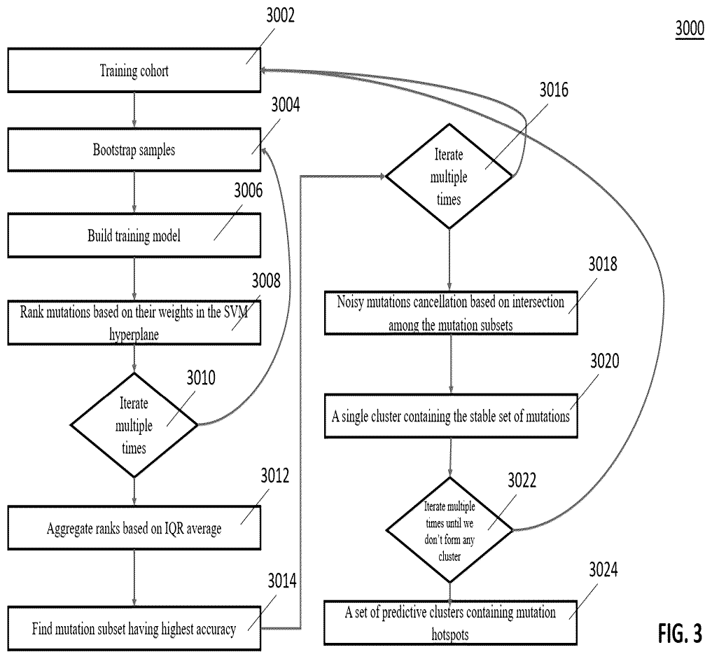

As illustrated in FIG. 3, once a dataset D has been constructed, mutation clusters prediction may be performed by using a set of linear support vector machines (LSVMs) L. In step 3004, the dataset D may be bootstrapped by randomly choosing isolates with replacements to form a marginally distinct dataset D′. Next in step 3006, a training model may be built where each LSVM may be trained by employing a dataset D″ by randomly choosing a subset of samples (r=90% in our experiment) with replacement from dataset D′. I.e., D″ is a slightly reduced version of D′ where we randomly chose a subset of samples from D′ to construct D″. Consequently, one may be able to generate slightly different weights for each of the mutations for different LSVMs, where the weight generated or produced by a LSVM for a particular mutation is directly proportional to its importance (i.e., the more significant of the mutation, the more weight will be associated with that mutation), as illustrated in step 3008. The observation is that if a mutation is informative, the associated weights will not be significantly varied with slight changes in the dataset multiple times. On the contrary, the weights of non-informative mutation will be varied significantly with slight changes in the dataset. Subsequently, the weights may be normalized by employing their absolute values. The normalization may be performed for each of the LSVM produced weight vectors. In some embodiments, steps 3004 to 3008 may be repeated multiple times to generate weights for each of the mutations or mutation groups. Subsequently, the normalized weights (produced by the set of LSVM L) assigned to each mutation may be sorted and the average of the middle 50% of the weights can be taken (i.e., take the interquartile range (IQR) of weights and averaging them), as shown in step 3012, and the outliers may be discarded. The mutations can be then sorted in a non-decreasing order based on their average weights. To find a subset of discriminating mutations, the sorted mutations may be linearly searched using algorithms such as a variation of the sequential forward search (SFS) algorithm. As the mutations are sorted based on their importance, we can linearly search through these rank-ordered set to identify a subset of mutations having highest predictive power together. It works as follows. At first, we take first top 5 mutations and compute their Matthews correlation coefficient (MCC) using 10-fold cross validation. Then we proceed with top 10 mutations and compute MCC as stated above. We iterate the same procedure for the rest (i.e., top 15, top 20, . . . , top N) mutations and pick the one among them having the highest MCC.MCC is mathematically defined as follows:

$$MCC = \frac{T P \times T N - F P \times F N}{\sqrt{( T P + F P ) ( T P + F N ) ( T N + F P ) ( T N + F N )}}$$

where TP, FN, FP, and TN are the number of True Positives, False Negatives, False Positives, and True Negatives, respectively. Once the MCC is calculated, one may choose to retain the top set having the highest MCC from the top variant sets, as illustrated in step 3014.

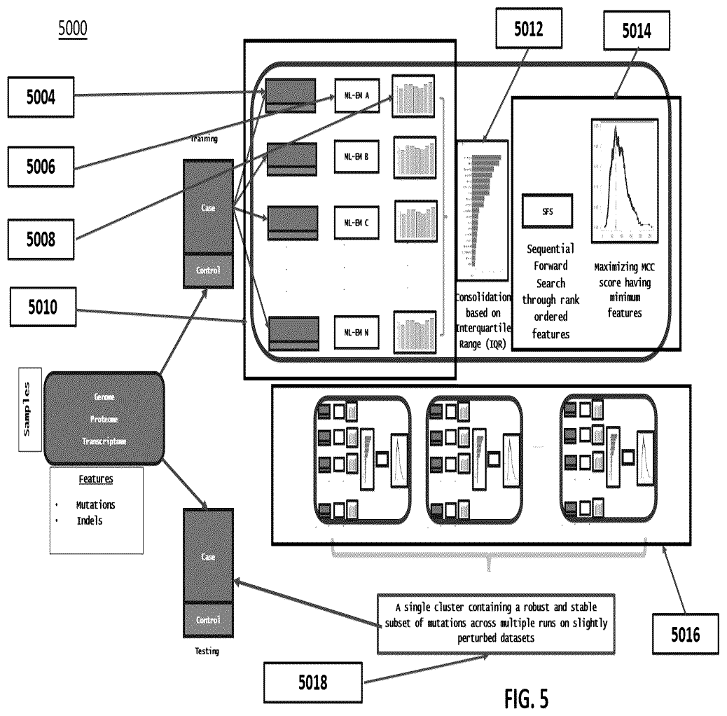

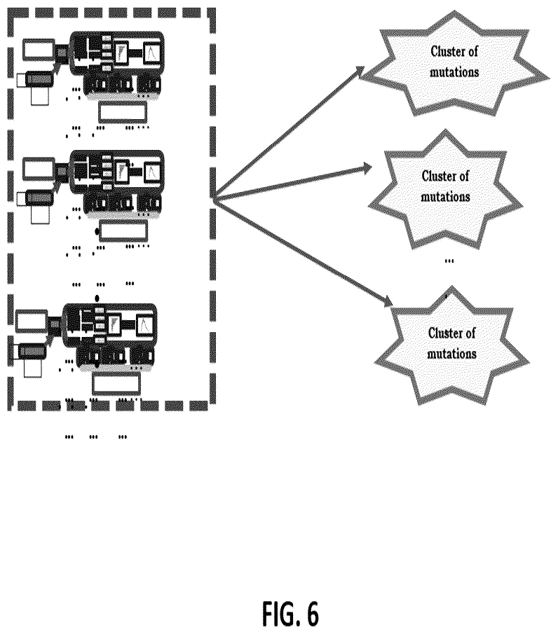

As illustrated in step 3016, this process may be iterated multiple times (e.g., n times) and from each iteration one may be able to generate a set of top mutation Sᵢ(where 1<=i<=n). Sometimes a set of mutations may be present in all such S₁ lists of mutations, then this set of mutations can be chosen to form a cluster c, as illustrated in FIG. 5. In this step 3016, this mutation cluster c may be removed from the dataset D and steps 3004 to step 3014 may be performed again. Subsequently, in steps 3018 and 3020, mutations may be discovered from all the subsets. For example: for p iterations, and from each iteration, a set of top mutation subset S₁ having highest MCC in the corresponding i^{th} iteration where 1≤i≤p may be discovered. Then a set of mutations S may be flagged in all such S₁ lists of mutations, i.e., S={S₁ ∩ S₂ ∩ S₃ ∩ S₄ ∩ . . . ∩ Sₚ}. Extracting the common mutations across different runs on slightly different datasets further eliminates probable noisy and unstable mutations from consideration. Now, this set of mutations forms a cluster C₁. This process may be repeated multiple times until no more clusters can be found, as illustrated in step 3022. At the end, in step 3024, a resulting set of clusters C containing a set of robust and stable mutations, as illustrated in FIG. 6.

FIG. 5 is a flow chart illustrating a similar process 5000 presented in FIG. 3. Referring now to FIG. 5, in block 5004: after a training cohort been bootstrapped, one may proceed to build a training model based on training a Linear SVM, as illustrated in block 5006. Subsequently, in block 5008, from the training model one may be able to generate weights for each mutation, where the weights directly proportion to its importance. As illustrated in block 5010, blocks 5004-5008 may be repeated multiple times to generate a set of weights for each of the mutations. Then one may average the weights of each mutation (i.e., aggregation) and rank the mutations based on their average weights (i.e., block 5012). Followed by block 5014, where the mutation subset among all the mutations having highest MCC may be identified based on a variation of Sequential Forward Search algorithm (SFS). For example: As the mutations are sorted based on their importance, one may linearly search through these rank-ordered set to identify a subset of mutations having highest predictive power together. In some embodiments, one may firstly take the top 5 mutations and compute their Matthews correlation coefficient (MCC) using the 10-fold cross validation. Then proceed with the top 10 mutations and compute MCC as stated above. The same procedure may be repeated for the rest (i.e., top 15, top 20, . . . , top N) of the mutations and the having the highest MCC may be chosen. Subsequently, in block 5016, one may iterate the entire procedure 5004-5014 multiple times and generate a subset of mutations each time having the highest predictive power by computing MCC. Then as illustrated in block 5018: one can identify those mutations that are found across all the subsets. For example: for p iterations, and in each iteration, one may discover a set of top mutation subset S₁ having highest MCC in the corresponding i^{th} iteration where 1≤i≤p. One then may be able to identify a set of mutations S that were found in all such S₁ lists of mutations, i.e., S={S₁ ∩ S₂ ∩ S₃ ∩ S₄ ∩ . . . ∩ Sₚ}. Extracting the common mutations across different runs on slightly different datasets further eliminate probable noisy and unstable mutations from consideration. Now, this set of mutations forms a cluster C₁. Subsequently, one may remove the mutations in C₁ from the training cohort and execute the entire procedure as stated above and repeat this procedure multiple times until no more predictive clusters can be generated. As such, a set of clusters C={C₁, C₂, C₃, . . . }containing a set of robust and stable mutations may be obtained at this point.

In some embodiments, the predictive power of the resulting clusters C may be assessed using a test dataset. In practice, one may firstly construct a training model based on the training isolates with selected mutations in each cluster. Then the train model may be validated using isolates from this completely previously “unseen” test dataset. I.e., we take each sample from test dataset and predict the phenotypic status of that sample using the training model. As we know the true phenotypic status of each isolate contained in test dataset a priory, we can assess the predictive power of our trained model by employing various kinds of binary classification accuracy measures such as the F1 measure, Youden's index, Matthews correlation coefficient (MCC), diagnostic odds ratio (DOR) etc. And clusters in C with MCC values exceeding 0.7 may be collected as output.

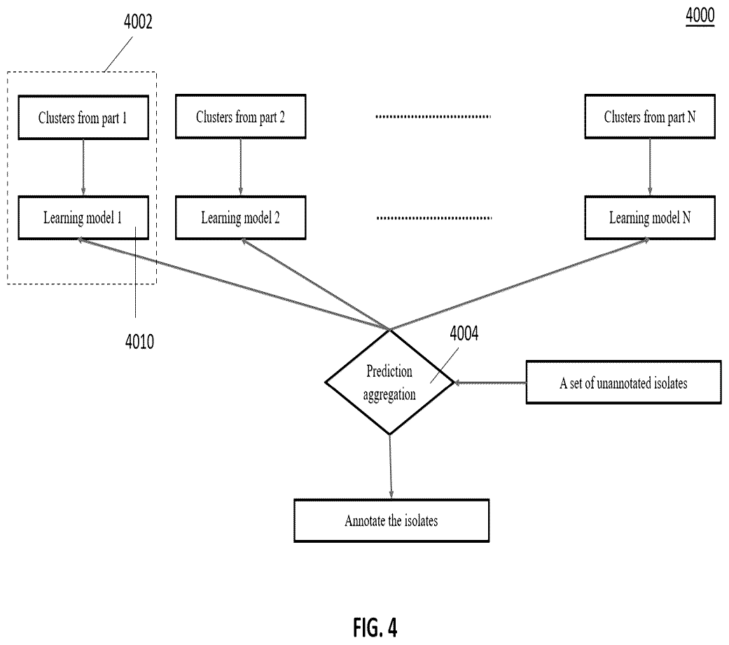

In some other embodiments, the amount of genome samples may become too large to be processed efficiently. In this case, a distributed learning framework 4000 may be adopted to process the samples. For example, referring now to FIG. 4, firstly, a relatively large dataset may be sampled into a set of disjoint datasets D_{dis}. Where for each dataset D in D_{dis}, an iterative mutation cluster prediction or IMCP algorithm similar to the one described above may be utilized to process the datasets in parallel, as illustrated in block 4002. Subsequently, the mutations in each cluster may be combined for each dataset D and may be used to construct a learning model based on those mutations using training examples. As a result, |D_{dis}| number of such learning models 4010 may be generated from the process. In some embodiments, in step 4004, all the models may be aggregated to predict the phenotypic status of an isolate. For example, let the set of learning models be x. Now assume a subset of learning models y in x predict a specific phenotypic status (such as, VOC) of an isolate of interest. We assign an ensemble probability P=|y|/|x| as the prediction probability of this isolate being of interest to a specific phenotypic status. For the purpose of illustrating the working principles presented herein, assume using a 10 learning models, and 5/10 models predict an isolate to be VOC. Then the resulting probability P=5/10=0.5 which states that there is a 50% chance that the particular isolate of interest will be a VOC, as illustrated in FIG. 6.

In practice, statistical models may be constructed to assess the effectiveness of the distributed learning framework outlined in FIG. 6. For example, a statistical model may include an entirely separated testing cohort T and a set of learning models L found from the distributed learning framework. Then the learning models L may be subsampled and aggregated to predict the phenotypic status of each sample in T. Because the actual phenotypic status of the samples in T is known, a confusion matrix and various types of performance scores may be generated to evaluate the distributed learning framework, performance scores such as the diagnostic odds ratio (DOR), where DOR is defined as the ratio of the odds that the patient tests positive with respect to the odds of testing positive being healthy. A DOR may be mathematically defined using the confusion matrix as follows:

$$Diagnostic odds ratio , D O R = \frac{T P / F N}{F P / T N} = \frac{T P / F P}{F N / T N} = \frac{T P \cdot T N}{F P \cdot F N}$$

This process may be repeated multiple times (i.e., subsampling the learning models and aggregating the predicted phenotypic status of each sample in T) and the corresponding DORs may be recorded. The DORs may be further transformed into log of DORs (LDORs) where LDOR is normally distributed, and one may be able to compute the statistical significance of the learning framework along with a confidence interval.

In some embodiments, the prediction methods presented herein may be further evaluated using two contrasting sets of samples from the GISAID SARS-CoV-2 database (https://www.gisaid.org/). For example, a Cases set may be constructed using samples with known positive phenotypic status (such as VOC, VOI, or dominant lineage), and a Controls set may be constructed with samples with negative phenotypic status (e.g., samples with non-VOC, non-VOI, non-VUM or non-dominant lineage). Furthermore, the GISAID SARS-CoV-2 genome-wide protein mutation dataset may be used to evaluate the validity of the prediction method presented herein. In some embodiments, the effectiveness of the prediction method or algorithms may be measured using performance metrics such as the Youden's index, the Matthews correlation coefficient (MCC), the Diagnostic odds ratio (DOR), and/or the F1 score. As such, the performance of the prediction algorithms presented herein may be formulated as 2-class classification problems. For examples, to predict if an isolate may be a VOC, one may firstly formulate a set of positive examples include isolates already announced as VOC, and then a set of negative examples with isolates not annotated as VOC, VOI, nor VUM. In one example, 4,000 isolates are chosen using the representative selection algorithm outlined in FIG. 1, where 50% of these 4,000 isolates are positive examples and the rest are negative examples. These 4,000 examples constitute the training dataset. Furthermore, a test dataset containing 1,500 samples may be constructed, of which 50% are positives samples and the rest are negatives samples. These 2 datasets are mutually exclusive, meaning, they don't have any overlap samples among them. When the iterative cluster prediction algorithm is executed on the training dataset, 10 mutation clusters were found. Subsequently, the accuracy of each mutation cluster may be assessed by employing the test dataset, for example, by taking mutations from each cluster and build a learning model based on the training data and subsequently, assess the trained model by using the test dataset. The performance evaluations are illustrated in Table 1. As shown in Table 1, 4 clusters with 61 mutations have a MCC>=0.7. A learning model constructed using all 61 mutations from these 4 clusters will be able to predict whether an isolate is a VOC with great accuracy.

TABLE 1
Prediction of variants of concern (VOCs).

| | | Confusion Matrix | | | | Performance Metrics | | | |
|---|---|---|---|---|---|---|---|---|---|
| Cluster | Mutations | TP | FP | TN | FN | F1 | Youden | MCC | DOR |
| 1 | 15 | 740 | 6 | 744 | 10 | 0.99 | 0.98 | 0.98 | 9176 |
| 2 | 30 | 724 | 17 | 733 | 26 | 0.97 | 0.94 | 0.94 | 1200.66 |
| 3 | 4 | 604 | 3 | 747 | 146 | 0.89 | 0.8 | 0.82 | 1030.11 |
| 4 | 12 | 508 | 8 | 742 | 242 | 0.8 | 0.67 | 0.7 | 194.7 |
| All | 61 | 748 | 0 | 750 | 2 | 0.999 | 0.997 | 0.997 | Infinity |

In some embodiments, to evaluate the robustness of the algorithms presented herein with respect to noise, instead of picking representatives, one may instead randomly select training and testing examples from samples having is_complete and is_high_coverage flags marked as true. For example, one training dataset may be constructed to include 8,000 samples where 50% of the samples are positives and the rest are negative. In addition, a test dataset may be constructed to include 3,000 samples where 50% of the samples are positives and the rest are negatives. These 2 datasets are mutually exclusive, meaning, they don't have any duplicate samples between them. When an iterative cluster prediction algorithm similar to the algorithms presented herein is executed on the training dataset 8 clusters of mutations were discovered. The accuracy of each cluster may be assessed by employing the test dataset, for example, taking mutations from each cluster and build a learning model based on the training examples and subsequently, assess the learning model by using the test dataset. The performance evaluations are shown in Table 2. As shown in Table 2, three clusters with total of 49 mutations have a MCC>=0.7. A learning model constructed using all 49 mutations from these three clusters will be able to predict whether an isolate is a VOC/VOI with great accuracy.

TABLE 2
Prediction of variants of concern (VOCs) and variants of interest (VOIs).

| | | Confusion Matrix | | | | Performance Metrics | | | |
|---|---|---|---|---|---|---|---|---|---|
| Cluster | Mutations | TP | FP | TN | FN | F1 | Youden | MCC | DOR |
| 1 | 22 | 1424 | 21 | 1479 | 76 | 0.967 | 0.935 | 0.934 | 1319.609 |
| 2 | 23 | 1485 | 55 | 1445 | 15 | 0.977 | 0.953 | 0.954 | 2601 |
| 3 | 4 | 1390 | 114 | 1386 | 110 | 0.925 | 0.851 | 0.851 | 153.6316 |
| All | 49 | 1462 | 16 | 1484 | 38 | 0.98 | 0.964 | 0.964 | 3568.432 |

In some embodiments, the subject matter presented herein may be used to predict VOCs before they are announced by the CDC. For example, a training dataset may be constructed to include 4,000 high quality isolates, where 2,000 high quality isolates may be randomly selected and annotated as VOCs one month prior the announcement of a particular variant being VOC. In practice, if one wanted to predict whether the covid-19's Alpha variant would be a VOC one month prior a CDC announcement, then the Alpha isolates will not be considered in finding the mutation clusters (i.e., the training dataset will not contain any Alpha variant's genomes). In addition, another 2,000 high quality isolates may be randomly selected and not annotated as VOI, VOC, or VUM one month prior a CDC announcement. A test dataset may be constructed to have 4,000 isolates where 2,000 high quality isolates of a particular variant (for instance, Alpha) may be randomly picked, and another 2,000 high quality isolates not in training examples and not annotated either as VOI, VOC, or VUM is also chosen. The performance evaluations are shown in Table 3, where the most predictive group of mutations is used to infer the status of VOCs. As illustrated in Table 3, 80% of all VOCs (except Delta) were predicted at least one month ahead of a COO announcement. In particular, Alpha, Beta, and Gamma variants were predicted more than 8 months ahead of a CDC announcement.

TABLE 3
Prediction of VOCs at least 1 month before the announcement.

| | | Earliest | | | | | | | | |
|---|---|---|---|---|---|---|---|---|---|---|
| | Date of | date of | Confusion Matrix | | | | Performance Metrics | | | |
| Variant | designation | prediction | TP | FP | TN | FN | MCC | Youden | F1 | DOR |
| Alpha | 29 Dec. 2020 | 11 Mar. 2020 | 1995 | 0 | 1993 | 5 | 0.997 | 0.998 | 0.999 | Infinity |
| Beta | 29 Dec. 2020 | 17 Feb. 2020 | 343 | 2 | 342 | 1 | 0.991 | 0.991 | 0.996 | 58653 |
| Gamma | 29 Dec. 2020 | 22 Apr. 2020 | 12 | 7 | 1989 | 0 | 0.793 | 0.996 | 0.77 | Infinity |
| Omicron | 30 Nov. 2021 | 1 Nov. 2021 | 1943 | 33 | 1966 | 57 | 0.955 | 0.955 | 0.977 | 2030.802 |

Furthermore, the subject matter presented herein may be used to provide probable dominant lineage prediction without the need for time-course data The present subject matter presents methods to predict computationally if a newly emerged variant will be dominant by learning from existing data. In one embodiment, two contrasting groups are firstly defined, a dominant group and a non-dominant group, and Covid-19's Omicron lineage is used herein as an example for the purpose of illustrating the working principles. In this setup, the dominant lineage may be past dominant lineages announced by CDC such as the BA.1 or BA.2 lineage; and the non-dominant lineages may be ones that were (1) Last observed on Apr. 30, 2022, and (2) incidences less than 20,000 worldwide. Overall, 1,259 high-quality isolates from 42 lineages/sub-lineages were chosen. Subsequently, a learning model based on mutations residing in the highly predictive clusters is constructed. Then, a set of high-quality genomes (e. g., 20 genomes) may be selected from each lineage of Omicron variant and predict their binary phenotypic status (i.e., dominant or non-dominant). Scoring is measured as the fraction of genomes predicted as dominant in each cluster. Ensemble score is measured by taking the average of all such scores. Table 4 illustrates the outcomes of the approach with respect to lineages in Omicron variant. In this setup the BA.2 variant was used as the dominant lineage to infer future dominant lineages, and BA.4 and BA.5 are already established dominant lineages. According to GISAID time-course analyses, most of the top 5 lineages predicted by the present algorithms are currently dominant lineages.

TABLE 4
Prediction of next dominant lineages within
Omicron variant without any time-course data.

| Lineage | Ensemble Score |
|---|---|
| BA.2.33 | 1 |
| BA.2.13 | 1 |
| BA.2.9 | 0.99 |
| BA.2.12.1 | 0.98 |
| BA.2.30 | 0.96 |
| BA.2.23 | 0.96 |
| BA.4 | 0.93 |
| BA.5 | 0.91 |
| XN | 0.9 |
| BA.2.18 | 0.88 |
| XH | 0.7 |
| XE | 0.66 |
| XJ | 0.6 |
| BA.3 | 0.115 |

It should be appreciated that the subject matter presented herein provides methods and algorithms for detecting mutation hotpots (i.e., mutation clusters) across the entire genomes. Furthermore, the methods and algorithms presented herein do not depend on time-course events. As such, one may infer VOCs and dominant lineages ahead of the time. As mentioned above, using the methods and algorithms presented herein enables one the ability to predict the Alpha, Beta, or Gamma variants eight plus months ahead of the CDC announcements.

In practice, an output generated by algorithms in accordance with the subject matter presented herein may be used to produce vaccines. Vaccines that may be designed to prevent infection against a particular strain of viral mutation. For example, the algorithm may be used to produce an optimized nucleic acid, such as an mRNA, that encodes a polypeptide antigen. The mRNA and/or the polypeptide antigen may be produced to be included in an immunogenic composition such as a vaccine. In some other embodiments, output generated by algorithms according to the subject matter presented herein may also be used to produce anti-viral drugs, where circulating strain may be used to produce drugs designed to target that strain, to target those associated conserved regions.

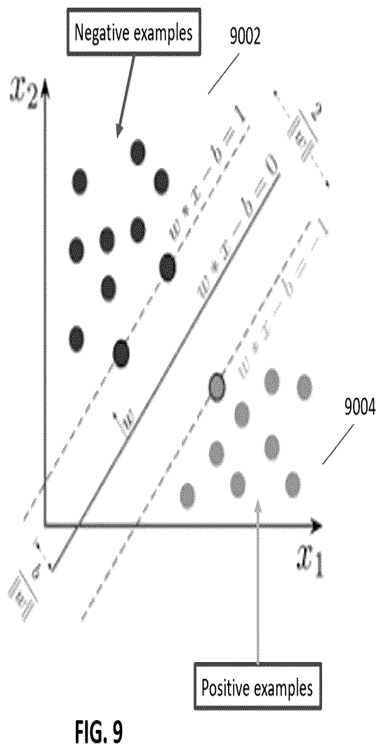

In some embodiments, referring now to FIG. 9, assuming one have at least 1000 isolates from a new variant just emerged, these new isolates may be treated as positive examples 9002, and the non-(VOC+VOI+VUM) set may be used as the negative examples 9004. Applying the machine learning classifiers and algorithms presented herein, one can successfully deduce that new variant will be VOC with a high level of confidence, as illustrated in FIG. 9. As such, the subject matter presented herein may only need a handful of high-quality genomes (for instance ˜1,000 positive and negative instances each) to elucidate the entire landscape of SARS-CoV-2 mutations and distinguish between VOCs and non-VOCs.

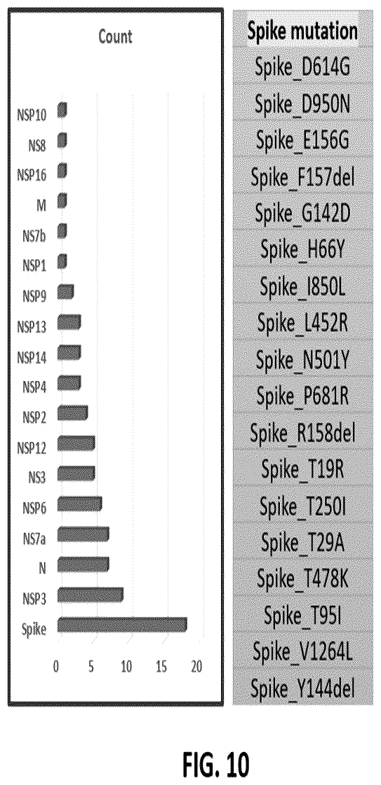

In one example, referring now to FIG. 10, 1,000 random samples from the SARS-DoV-2 Delta are used as positive examples, 1,000 random samples from non-(VOC+VOI+VUM) are used as negative examples, and a test dataset contains 200+200 random samples from Delta and non-(VOC+VOI+VUM) set are used. Applying the subject matter disclosed herein (e.g., Machine learning classifiers and algorithms), 8 clusters were identified (78 genome-wide mutations in total) that can perfectly separate Delta from non-(VOC+VOI+VUM) isolates.

Many different arrangements of the various components depicted, as well as components not shown, are possible without departing from the scope of the claims below. Embodiments of the disclosure have been described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

What is claimed is:

1. A method for screening genetic mutations to predict vaccine composition, the method comprising:
selecting a plurality of genome samples;
identifying genomic isolates with phenotypic statuses from the selected genome samples by training at least one linear support vector machine with the genome samples; and
assessing the identified genomic isolates using a performance metric.

2. The method of claim 1, wherein the step of selecting a plurality of genome samples comprises identifying a reference genome.

3. The method of claim 2, wherein the step of selecting a plurality of genome samples comprises collect a set of all k-mers from the sequence of the reference genome.

4. The method of claim 1, wherein the step of identifying genomic isolates with phenotypic statuses comprises producing statistical weights for genomic mutations.

5. The method of claim 6, wherein the step of identifying genomic isolates with phenotypic statuses comprises normalizing the produced statistical weights by calculating the weight's absolute values.

6. The method of claim 5, wherein the step of identifying genomic isolates with phenotypic statuses comprises averaging the produced statistical weights along the weight's interquartile range (IQR).

7. The method of claim 6 further comprising sorting the weights in a non-decreasing order based on their averaged values.

8. A method for screening genetic mutations to produce vaccines, the method comprising:
selecting a plurality of genome samples;
partitioning the plurality of genome samples into N groups, where N is an integer larger than 1;
identifying genomic isolates with phenotypic statuses from each of the N groups of genome samples by training at least one linear support vector machine with the genome samples, the identification of the isolates between each of the N groups of the genomic isolates performed in parallel; and
assessing the identified genomic isolates using a performance metric.

9. The method of claim 8, wherein the step of selecting a plurality of genome samples comprises identifying a reference genome.

10. The method of claim 9, wherein the step of selecting a plurality of genome samples comprises collect a set of all k-mers from the sequence of the reference genome.

11. The method of claim 8, wherein the step of identifying genomic isolates with phenotypic statuses comprises producing statistical weights for genomic mutations.

12. The method of claim 11, wherein the step of identifying genomic isolates with phenotypic statuses comprises normalizing the produced statistical weights by calculating the weight's absolute values.

13. The method of claim 12, wherein the step of identifying genomic isolates with phenotypic statuses comprises averaging the produced statistical weights along the weight's interquartile range (IQR).

14. A computerized system for screening genetic mutations for predicting vaccine composition, the system comprising:
one or more processors; and
a computer memory having computer-executable instructions stored thereon for performing operations when executed by one or more processors, the operations comprising:
selecting a plurality of genome samples;
identifying genomic isolates with phenotypic statuses from the selected genome samples by training at least one linear support vector machine with the genome samples;
assessing the identified genomic isolates using a performance metric; and
producing nucleic acid compositions using the identified genomic isolates to produce a vaccine.

15. The computerized system of claim 14, wherein the step of selecting a plurality of genome samples comprises collect a set of all k-mers from the sequence of the reference genome.

16. The computerized system of claim 14, wherein the step of identifying genomic isolates with phenotypic statuses comprises producing statistical weights for genomic mutations.

17. The computerized system of claim 16, wherein the step of identifying genomic isolates with phenotypic statuses comprises normalizing the produced statistical weights by calculating the weight's absolute values.

18. The computerized system of claim 17, wherein the step of identifying genomic isolates with phenotypic statuses comprises averaging the produced statistical weights along the weight's interquartile range (IQR).

19. The computerized system of claim 18, further comprising sorting the weights in a non-decreasing order based on their averaged values.

20. The computerized system of claim 20, wherein the performance metric is a Matthews Correlation Coefficient (MCC).
